(19) United States
(12) Patent Application Publication
(10) Pub. No.: US 2026/0151588 A1
(43) Pub. Date: Jun. 4, 2026

(54) URINARY CATHETER KITS

(71) Applicant: ConvaTec Limited, Flintshire (GB)

(72) Inventors: Alice Young, Chester (GB); Gavin Roberts, Swindon (GB)

(21) Appl. No.: 19/465,261

(22) Filed: Jan. 30, 2026

Related U.S. Application Data
(63) Continuation of application No. PCT/GB2024/051962, filed on Jul. 26, 2024.
(60) Provisional application No. 63/516,577, filed on Jul. 31, 2023.

(30) Foreign Application Priority Data
Sep. 8, 2023 (GB) 2313733.4

(51) Int. Cl. A61M 25/00

(52) U.S. Cl. CPC A61M 25/0017; A61M 2202/0014; A61M 2202/0496; A61M 2207/00; A61M 2210/1078

(57) ABSTRACT

The disclosure provides a urine collection bag comprising a cavity for containing urine, the bag comprising a trilayer laminate film, the film comprising in order: a first ethylene-vinyl acetate layer, a polyethylene layer, and a second ethylene-vinyl acetate layer.

### TECHNICAL FIELD OF THE DISCLOSURE

The present disclosure relates to urine collection bags and catheter sleeves comprising a laminate film material.

### Background to the Disclosure

Urinary catheterisation is a common medical procedure used for draining urine from patients who are unable to do so naturally. Typically, a catheter is inserted into the patient's urethra and connected to a urine collection bag, allowing for the collection and monitoring of urine output. In order to ensure patient comfort and hygiene, it is crucial to develop catheter kits with components, such as urine collection bags and catheter sleeves, that offer a range of beneficial features.

One important consideration in designing catheter kit components is the choice of materials. Conventional urine collection bags and catheter sleeves are often made of thick plastic materials, which can result in bulkiness, increased weight, and discomfort for the patient. Additionally, the use of large amounts of plastic contributes to environmental concerns. Conventional materials are often known to suffer from further disadvantages including that they may experience discolouration or degradation when exposed to sterilisation methods such as steam or chemical agents. Further, conventional materials are known to produce disruptive sounds during movement, such as rustling sounds, reducing patient comfort and privacy during the catheterisation process.

There exists a need for new and improved materials for catheterisation applications, which overcome or mitigate one or more problems of the prior art mentioned above.

It is an aim of embodiments of the present disclosure to overcome or mitigate at least one problem of the prior art, whether expressly described herein or not.

### SUMMARY OF THE DISCLOSURE

According to a first aspect of the disclosure, there is provided a urine collection bag comprising a cavity for containing urine, the bag comprising a trilayer laminate film, the film comprising in order: a first ethylene-vinyl acetate layer, a polyethylene layer, and a second ethylene-vinyl acetate layer.

Such a trilayer laminate film provides several advantages over traditional materials. The layer combination employed in the trilayer film confers exceptional strength to the material, even in the case of thin films. As such, even a thin layer of the trilayer film may be successfully used in construction of catheter kit components, thus significantly reducing the amount of plastic required in manufacture. This reduction in plastic usage helps minimise waste and contributes to environmental sustainability. The film is also lightweight without any compromise to strength, and so allows for development of lightweight urine collection bags that can still hold large amounts of urine effectively. The film is also relatively quiet when handled or in transport and does not produce significant rustling or other disruptive sounds during movement, which enhances patient comfort and privacy. Additionally, the trilayer laminate film is also believed to provide excellent resistance to discolouration when subjected to sterilisation processes. Sterilisation is a critical step to maintain the hygiene and safety of catheter kit components and the film is believed to preserve its appearance and integrity even after repeated sterilisation cycles.

In some embodiments, at least 50 wt. % of the bag may comprise the trilayer film, or at least 60, 70, 80, 90, or at least 96 wt. % of the bag may comprise the trilayer film. Preferably, the bag may be made entirely of the trilayer film.

In some embodiments, the cavity of the bag is defined by the trilayer film.

In some embodiments, the bag comprises a front wall and a rear wall, wherein the front and rear walls are sealed together to form the cavity of the bag therebetween, and wherein the front and rear wall comprise the trilayer film.

In some embodiments, the front and rear walls are sealed together at their peripheries to form the cavity. The front and rear walls may be welded together at their peripheries to form the cavity. The weld may pass through all the layers of the films of the front and rear walls.

In some embodiments, the front or rear wall comprises an inlet for receiving urine.

In some embodiments, the bag comprises an attachment element for attaching the bag to a urinary catheter. In some embodiments, the attachment element comprises a mechanical fastening means. The attachment element may comprise a non-return valve to prevent flow of urine from the bag back into the catheter, in use.

The bag may be configured to hold at least 400 mL of urine, or at least 450, 500, 550, 600, 650, 700, or at least 750 mL of urine. The bag may be configured to hold between 500-2000 mL of urine, or between 750-1500 mL of urine.

In some embodiments, at least one layer of the film has a thickness of at least 2 microns, or at least 3, 4, 5, 6, 7, 8, 9 or at least 10 microns. In some embodiments, at least one layer of the film has a thickness of no greater than 80 microns, 75, 70, 65, 60, 55, 50, 45, or no greater than 40 microns. At least one layer of the film may have a thickness of between 10-40 microns, or between 15-35, or between 20-30 microns.

In some embodiments, at least one ethylene-vinyl acetate layer of the film has a thickness of at least 2 microns, or at least 3, 4, 5, 6, 7, 8, 9 or at least 10 microns. In some embodiments, at least one ethylene-vinyl acetate layer of the film has a thickness of no greater than 80 microns, 75, 70, 65, 60, 55, 50, 45, or no greater than 40 microns. At least one ethylene-vinyl acetate layer of the film may have a thickness of between 10-40 microns, or between 15-35, or between 20-30 microns. The first and/or second ethylene-vinyl acetate layer may have a thickness as described above.

In some embodiments, the polyethylene layer of the film has a thickness of at least 2 microns, or at least 3, 4, 5, 6, 7, 8, 9 or at least 10 microns. In some embodiments, the polyethylene layer of the film has a thickness of no greater than 80 microns, 75, 70, 65, 60, 55, 50, 45, or no greater than 40 microns. The polyethylene layer of the film may have a thickness of between 10-40 microns, or between 15-35, or between 20-35 microns, or between 25-35 microns.

In some embodiments, the first and second ethylene-vinyl acetate layers have a thickness of between 10-40 microns each; and the polyethylene layer has a thickness of between 10-40 microns, or between 15-35, or between 20-35 microns, or between 25-35 microns.

In some embodiments, the first and second ethylene-vinyl acetate layers have a thickness of between 15-35 microns each; and the polyethylene layer has a thickness of between 10-40 microns, or between 15-35, or between 20-35 microns, or between 25-35 microns.

In some embodiments, the first and second ethylene-vinyl acetate layers have a thickness of between 20-30 microns each; and the polyethylene layer has a thickness of between 10-40 microns, or between 15-35, or between 20-35 microns, or between 25-35 microns.

In some embodiments, the film has a total thickness of at least 40 microns, or at least 45, or at least 50 microns. The film may have a total thickness of no greater than 150 microns, or no greater than 145, 140, 135, 130, 125, 120, 115, 110, 105, or no greater than 100 microns. The film may have a total thickness of between 40-100 microns, or between 45-100, or between 50-100 microns, or between 55-95, or preferably between 60-90, 65-85, or preferably between 70-80 microns.

In some embodiments, the film may have a total thickness of between 40-95 microns, or between 45-90 microns.

In some embodiments, both the first and second ethylene-vinyl acetate layers of the film have the same thickness. The first and/or second ethylene-vinyl acetate layer may have the same thickness as the polyethylene layer. In some embodiments, all three layers of the trilayer film have the same thickness.

In other embodiments, the ethylene-vinyl acetate layers of the film have a different thickness to the polyethylene layer. The polyethylene layer may be thicker than the ethylene-vinyl acetate layers. In some embodiments, all three layers of the trilayer film have a different thickness.

The polyethylene layer may comprise polyethylene in a total amount of at least 80 wt. % of the polyethylene layer, or at least 85, 90, or at least 95 wt. % of the polyethylene layer. In some embodiments, the polyethylene layer is entirely made of polyethylene.

In some preferred embodiments, there is provided a urine collection bag comprising a cavity for containing urine, the bag comprising a trilayer laminate film, the film comprising in order: a first ethylene-vinyl acetate layer, a polyethylene layer, and a second ethylene-vinyl acetate layer, wherein the polyethylene layer of the film comprises polyethylene in a total amount of at least 80 wt. % of the polyethylene layer, and preferably the polyethylene layer is entirely made of polyethylene.

In some preferred embodiments, the polyethylene layer of the film comprises ethylene-vinyl acetate in a total amount of less than 10 wt. % of the polyethylene layer, or less than 5 wt. % of the polyethylene layer. In preferred embodiments, the polyethylene layer does not comprise ethylene-vinyl acetate.

In some preferred embodiments, there is provided a urine collection bag comprising a cavity for containing urine, the bag comprising a trilayer laminate film, the film comprising in order: a first ethylene-vinyl acetate layer, a polyethylene layer, and a second ethylene-vinyl acetate layer, wherein the polyethylene layer of the film comprises ethylene-vinyl acetate in a total amount of less than 10 wt. % of the polyethylene layer, and preferably the polyethylene layer does not comprise ethylene-vinyl acetate

At least one ethylene-vinyl acetate layer may comprise ethylene-vinyl acetate in a total amount of at least 80 wt. % of the ethylene-vinyl acetate layer, or at least 85, 90, or at least 95 wt. % of the ethylene-vinyl acetate layer. In some embodiments, the ethylene-vinyl acetate layer is entirely made of ethylene-vinyl acetate.

In some preferred embodiments, there is provided a urine collection bag comprising a cavity for containing urine, the bag comprising a trilayer laminate film, the film comprising in order: a first ethylene-vinyl acetate layer, a polyethylene layer, and a second ethylene-vinyl acetate layer, wherein at least one ethylene-vinyl acetate layer of the film comprises ethylene-vinyl acetate in a total amount of at least 95 wt. % of the ethylene-vinyl acetate layer, and preferably the at least one ethylene-vinyl acetate layer is entirely made of ethylene-vinyl acetate.

In some preferred embodiments, at least one ethylene-vinyl acetate layer of the film comprises polyethylene in a total amount of less than 10 wt. % of the ethylene-vinyl acetate layer, or less than 5 wt. % of the ethylene-vinyl acetate layer. In preferred embodiments, at least one ethylene-vinyl acetate layer and preferably both the first and second ethylene-vinyl acetate layers does not comprise polyethylene.

In some preferred embodiments, there is provided a urine collection bag comprising a cavity for containing urine, the bag comprising a trilayer laminate film, the film comprising in order: a first ethylene-vinyl acetate layer, a polyethylene layer, and a second ethylene-vinyl acetate layer, wherein at least one ethylene-vinyl acetate layer of the film comprises polyethylene in a total amount of less than 10 wt. % of the ethylene-vinyl acetate layer, and preferably the at least one ethylene-vinyl acetate layer does not comprise polyethylene.

In some preferred embodiments, the first and second ethylene-vinyl acetate layers of the film are each entirely made of ethylene-vinyl acetate, and the polyethylene layer of the film is entirely made of polyethylene.

In some preferred embodiments, there is provided a urine collection bag comprising a cavity for containing urine, the bag comprising a trilayer laminate film, the film comprising in order: a first ethylene-vinyl acetate layer, a polyethylene layer, and a second ethylene-vinyl acetate layer, wherein the first and second ethylene-vinyl acetate layers of the film are each entirely made of ethylene-vinyl acetate, and the polyethylene layer of the film is entirely made of polyethylene.

It has been found that providing a layered film structure in which ethylene-vinyl acetate is largely separated from polyethylene, and in which both materials are present in discrete layers with the polyethylene layer sandwiched between ethylene-vinyl acetate, allows for a film that not only has excellent strength, but which is also quiet when handled and produces minimal rustling or other disruptive sounds. In addition, such a film structure provides excellent resistance to discolouration even when the film is subjected to multiple sterilisation processes.

In some embodiments, the film comprises polyethylene in a total amount of at least 2 wt. %, or at least 3, 4, or at least 5 wt. %. In some embodiments, the film comprises polyethylene in a total amount of no greater than 80 wt. %, 75, or no greater 70 wt. %, or no greater than 60, 50, 40, 30, 20, or no greater than 15 wt. %.

In some embodiments, the film comprises polyethylene in a total amount of between 5-70 wt. %, or between 10-70, or between 15-70, or between 20-70, or between 30-60, or between 30-50 wt. %. In other embodiments, the film comprises polyethylene in a total amount of between 2-20 wt. %, or between 5-15, or between 7-13 wt. %.

In some preferred embodiments, the film comprises polyethylene in a total amount of between 5-50 wt. %, or preferably between 10-45 wt. %.

In some preferred embodiments, there is provided a urine collection bag comprising a cavity for containing urine, the bag comprising a trilayer laminate film, the film comprising in order: a first ethylene-vinyl acetate layer, a polyethylene layer, and a second ethylene-vinyl acetate layer, wherein the film comprises polyethylene in a total amount of between 5-50 wt. %, or preferably between 10-45 wt. %.

In some embodiments, the film comprises ethylene-vinyl acetate in a total amount of at least 2 wt. %, or at least 3, 4, 5, 10, 15, 20, or at least 25 wt. %. In some embodiments, the film comprises ethylene-vinyl acetate in a total amount of no greater than 95 wt. %, or no greater than 90, 85, 80, 75, or no greater 70 wt. %, or no greater than 60, 50, 40, 30, 20, or no greater than 15 wt. %.

In some embodiments, the film comprises ethylene-vinyl acetate in a total amount of between 5-70 wt. %, or between 10-70, or between 15-70, or between 20-70, or between 30-60, or between 30-50 wt. %. In other embodiments, the film comprises ethylene-vinyl acetate in a total amount of between 80-98 wt. %, or between 75-95, or between 87-93 wt. %.

In some preferred embodiments, the film comprises ethylene-vinyl acetate in a total amount of between 50-95 wt. %, or preferably between 55-90 wt. %.

In some preferred embodiments, there is provided a urine collection bag comprising a cavity for containing urine, the bag comprising a trilayer laminate film, the film comprising in order: a first ethylene-vinyl acetate layer, a polyethylene layer, and a second ethylene-vinyl acetate layer, wherein the film comprises ethylene-vinyl acetate in a total amount of between 50-95 wt. %, or preferably between 55-90 wt. %.

In some embodiments, the film comprises polyethylene in a total amount of between 10-70 wt. % and ethylene-vinyl acetate in a total amount of between 30-90 wt. %. The film may comprise polyethylene in a total amount of between 20-60 wt. % and ethylene-vinyl acetate in a total amount of between 40-80 wt. %. The film may comprise polyethylene in a total amount of between 30-50 wt. % and ethylene-vinyl acetate in a total amount of between 50-70 wt. %.

In other embodiments, the film comprises polyethylene in a total amount of between 2-20 wt. % and the film comprises ethylene-vinyl acetate in a total amount of between 80-98 wt. %. The film may comprise polyethylene in a total amount of between 5-15 wt. % and the film may comprise ethylene-vinyl acetate in a total amount of between 85-95 wt. %. The film may comprise polyethylene in a total amount of between 7-13 wt. % and the film may comprise ethylene-vinyl acetate in a total amount of between 87-93 wt. %.

In some embodiments, the film has a total thickness of between 50-100 microns; and the film comprises polyethylene in a total amount of between 5-70 wt. %, or between 10-70, or between 15-70, or between 20-70, or between 30-60, or between 30-50 wt. %. In other embodiments, the film may have a total thickness of between 50-100 microns; and the film may comprise polyethylene in a total amount of between 2-20 wt. %, or between 5-15, or between 7-13 wt. %.

In some embodiments, the film has a total thickness of between 60-90 microns; and the film comprises polyethylene in a total amount of between 5-70 wt. %, or between 10-70, or between 15-70, or between 20-70, or between 30-60, or between 30-50 wt. %. In other embodiments, the film may have a total thickness of between 60-90 microns; and the film may comprise polyethylene in a total amount of between 2-20 wt. %, or between 5-15, or between 7-13 wt. %.

In some embodiments, the film has a total thickness of between 70-80 microns; and the film comprises polyethylene in a total amount of between 5-70 wt. %, or between 10-70, or between 15-70, or between 20-70, or between 30-60, or between 30-50 wt. %. In other embodiments, the film may have a total thickness of between 70-80 microns; and the film may comprise polyethylene in a total amount of between 2-20 wt. %, or between 5-15, or between 7-13 wt. %.

In some embodiments, the polyethylene layer comprises low-density polyethylene. The polyethylene layer may comprise linear low-density polyethylene.

In some embodiments, the ethylene-vinyl acetate of the first and/or second ethylene-vinyl acetate layer may have a vinyl acetate content of between 10-20% of the copolymer, or between 15-20% of the copolymer.

In some preferred embodiments, the trilayer film is symmetrical. In other embodiments, the first ethylene-vinyl acetate layer may be thicker or thinner than the second ethylene-vinyl acetate layer.

In some embodiments, the trilayer film is a cast film. Alternatively, the trilayer film may be a blown film.

According to a second aspect of the disclosure, there is provided a catheter sleeve comprising a trilayer laminate film comprising in order: a first ethylene-vinyl acetate layer, a polyethylene layer, and a second ethylene-vinyl acetate layer.

The trilayer laminate film of the second aspect of the disclosure may be the trilayer film of the first aspect of the disclosure above. Statements of disclosure above relating to the trilayer film of the first aspect of the disclosure may be applied mutatis mutandis to the second aspect of the disclosure. Statements of disclosure below for the second aspect of the disclosure may also be applied mutatis mutandis to the other aspects of the disclosure.

In some embodiments, the sleeve may comprise a tubular body comprising the trilayer film and which tubular body comprises a hollow lumen extending longitudinally therethrough. The hollow lumen may preferably be adapted to receive a catheter, in use.

In some embodiments, at least 50 wt. % of the sleeve may comprise the trilayer film, or at least 60, 70, 80, 90, or at least 96 wt. % of the sleeve may comprise the trilayer film. Preferably, the sleeve may be made entirely of the trilayer film.

Film thicknesses for the sleeve of the second aspect of the disclosure may be as described for the bag of the first aspect of the disclosure. Alternatively, the film of the sleeve of the second aspect of the disclosure may have a different thickness to the film of the bag of the first aspect of the disclosure. The film of the sleeve of the second aspect of the disclosure may be thinner than the film of the bag of the first aspect of the disclosure.

In some embodiments, at least one layer of the film has a thickness of at least 1 micron, or at least 2, 3, 4, or at least 5 microns. In some embodiments, at least one layer of the film has a thickness of no greater than 50 microns, or no greater than 45, 40, 35, or no greater than 30 microns. At least one layer of the film may have a thickness of between 5-30 microns, or between 10-25, or between 15-20 microns.

In some embodiments, at least one ethylene-vinyl acetate layer of the film has a thickness of at least 1 micron, or at least 2, 3, 4, or at least 5 microns. In some embodiments, at least one ethylene-vinyl acetate layer of the film has a thickness of no greater than 50 microns, or no greater than 45, 40, 35, or no greater than 30 microns. At least one ethylene-vinyl acetate layer of the film may have a thickness of between 5-30 microns, or between 10-25, or between 10-20 microns. The first and/or second ethylene-vinyl acetate layer may have a thickness as described above.

In some embodiments, the polyethylene layer of the film has a thickness of at least 1 micron, or at least 2, 3, 4, or at least 5 microns. In some embodiments, the polyethylene layer of the film has a thickness of no greater than 60 microns, or no greater than 55, 50, 45, 40, 35, or no greater than 30 microns. The polyethylene layer of the film may have a thickness of between 5-30 microns, or between 10-25, or between 15-25 microns.

In some embodiments, the film has a total thickness of at least 20 microns, or at least 25, 30, 35, or at least 40 microns. The film may have a total thickness of no greater than 100 microns, or no greater than 90, 80, or no greater than 70 microns. The film may have a total thickness of between 40-95 microns, or between 40-90 microns, or between 40-70 microns, or between 45-65, 45-60, or preferably between 45-55 microns.

In some embodiments, the film has a total thickness of between 40-95 microns, or between 45-90 microns.

In some embodiments, both the first and second ethylene-vinyl acetate layers of the film have the same thickness. The first and/or second ethylene-vinyl acetate layer may have the same thickness as the polyethylene layer. In some embodiments, all three layers of the trilayer film have the same thickness.

In other embodiments, the ethylene-vinyl acetate layers of the film have a different thickness to the polyethylene layer. The polyethylene layer may be thicker than the ethylene-vinyl acetate layers. In some embodiments, all three layers of the trilayer film have a different thickness.

In some embodiments, the film has a total thickness of between 40-70 microns; and the film comprises polyethylene in a total amount of between 5-70 wt. %, or between 10-70, or between 15-70, or between 20-70, or between 30-60, or between 30-50 wt. %. In other embodiments, the film may have a total thickness of between 40-70 microns; and the film may comprise polyethylene in a total amount of between 2-20 wt. %, or between 5-15, or between 7-13 wt. %.

In some embodiments, the film has a total thickness of between 45-65 microns; and the film comprises polyethylene in a total amount of between 5-70 wt. %, or between 10-70, or between 15-70, or between 20-70, or between 30-60, or between 30-50 wt. %. In other embodiments, the film may have a total thickness of between 45-65 microns; and the film may comprise polyethylene in a total amount of between 2-20 wt. %, or between 5-15, or between 7-13 wt. %.

In some embodiments, the film has a total thickness of between 45-60 microns; and the film comprises polyethylene in a total amount of between 5-70 wt. %, or between 10-70, or between 15-70, or between 20-70, or between 30-60, or between 30-50 wt. %. In other embodiments, the film may have a total thickness of between 45-60 microns; and the film may comprise polyethylene in a total amount of between 2-20 wt. %, or between 5-15, or between 7-13 wt. %.

In some embodiments, the film has a total thickness of between 45-55 microns; and the film comprises polyethylene in a total amount of between 5-70 wt. %, or between 10-70, or between 15-70, or between 20-70, or between 30-60, or between 30-50 wt. %. In other embodiments, the film may have a total thickness of between 45-55 microns; and the film may comprise polyethylene in a total amount of between 2-20 wt. %, or between 5-15, or between 7-13 wt. %.

In some preferred embodiments, there is provided a catheter sleeve comprising a trilayer laminate film comprising in order: a first ethylene-vinyl acetate layer, a polyethylene layer, and a second ethylene-vinyl acetate layer, wherein the polyethylene layer of the film comprises polyethylene in a total amount of at least 80 wt. % of the polyethylene layer, and preferably the polyethylene layer is entirely made of polyethylene.

In some preferred embodiments, there is provided a catheter sleeve comprising a trilayer laminate film comprising in order: a first ethylene-vinyl acetate layer, a polyethylene layer, and a second ethylene-vinyl acetate layer, wherein the polyethylene layer of the film comprises ethylene-vinyl acetate in a total amount of less than 10 wt. % of the polyethylene layer, and preferably the polyethylene layer does not comprise ethylene-vinyl acetate.

In some preferred embodiments, there is provided a catheter sleeve comprising a trilayer laminate film comprising in order: a first ethylene-vinyl acetate layer, a polyethylene layer, and a second ethylene-vinyl acetate layer, wherein at least one ethylene-vinyl acetate layer of the film comprises ethylene-vinyl acetate in a total amount of at least 95 wt. % of the ethylene-vinyl acetate layer, and preferably the at least one ethylene-vinyl acetate layer is entirely made of ethylene-vinyl acetate.

In some preferred embodiments, there is provided a catheter sleeve comprising a trilayer laminate film comprising in order: a first ethylene-vinyl acetate layer, a polyethylene layer, and a second ethylene-vinyl acetate layer, wherein at least one ethylene-vinyl acetate layer of the film comprises polyethylene in a total amount of less than 10 wt. % of the ethylene-vinyl acetate layer, and preferably the at least one ethylene-vinyl acetate layer does not comprise polyethylene.

In some preferred embodiments, there is provided a catheter sleeve comprising a trilayer laminate film comprising in order: a first ethylene-vinyl acetate layer, a polyethylene layer, and a second ethylene-vinyl acetate layer, wherein the first and second ethylene-vinyl acetate layers of the film are each entirely made of ethylene-vinyl acetate, and the polyethylene layer of the film is entirely made of polyethylene.

In some preferred embodiments, there is provided a catheter sleeve comprising a trilayer laminate film comprising in order: a first ethylene-vinyl acetate layer, a polyethylene layer, and a second ethylene-vinyl acetate layer, wherein the film comprises polyethylene in a total amount of between 5-50 wt. %, or preferably between 10-45 wt. %.

In some preferred embodiments, there is provided a catheter sleeve comprising a trilayer laminate film comprising in order: a first ethylene-vinyl acetate layer, a polyethylene layer, and a second ethylene-vinyl acetate layer, wherein the film comprises ethylene-vinyl acetate in a total amount of between 50-95 wt. %, or preferably between 55-90 wt. %.

According to third aspect of the disclosure, there is provided a urinary catheter kit comprising: a catheter comprising a proximal end for insertion into the body and a distal end; a urine collection bag comprising an inlet adapted for fluid communication with the catheter distal end; and a catheter sleeve configured to enclose at least part of the catheter length from the proximal end to the distal end, wherein the urine collection bag and catheter sleeve comprise a trilayer laminate film, the film comprising in order: a first ethylene-vinyl acetate layer, a polyethylene layer, and a second ethylene-vinyl acetate layer.

The urine collection bag of the third aspect of the disclosure may be the urine collection bag of the first aspect of the disclosure.

The sleeve of the third aspect of the disclosure may be the sleeve of the second aspect of the disclosure.

The trilayer film of the third aspect of the disclosure may preferably be the trilayer film of the first and/or second aspect of the disclosure.

Statements of disclosure above for the first and second aspects of the disclosure may also be applied mutatis mutandis to the third aspect of the disclosure.

The trilayer film of the sleeve may be identical to the trilayer film of the bag. Alternatively, the trilayer film of the sleeve may have a different film thickness to the trilayer film of the bag. The trilayer film of the sleeve may be thinner than the trilayer film of the bag. The trilayer film of the bag may have a film thickness as described in statements of disclosure for the first aspect of the disclosure above. The trilayer film of the sleeve may have a film thickness as described in statements of disclosure for the second aspect of the disclosure above.

In some embodiments, the sleeve may be configured to enclose at least 5% of the length of the catheter from the proximal end of the catheter to the distal end, or at least 10, 20, 30, 40 or at least 50% of the length of the catheter from the proximal end of the catheter to the distal end.

In some embodiments, the sleeve may be configured to enclose the entire length of the catheter from the proximal end to the distal end. In some embodiments, the sleeve may not enclose the entire length of the catheter from the proximal end to the distal end. In such embodiments, the sleeve may be slidable along the entire length of the catheter, in use.

The catheter may be an intermittent catheter. Such a catheter is typically inserted into a body for short time periods, such as less than a day. Alternatively, the catheter may be an indwelling (Foley) catheter. Such a catheter is typically inserted and kept in a body for long periods of time, such as several days to months.

The catheter may be a single use catheter or a multiple use catheter.

In some embodiments, the catheter comprises a hollow tubular body, preferably a hollow polymeric tubular body. The hollow polymeric tubular body may comprise a base polymer.

In some embodiments, the catheter further comprises at least one additive, preferably at least one lubricious additive. Preferably, the hollow tubular body comprises at least one additive.

In some embodiments, at least one additive is a hydrophilic or amphiphilic additive.

In some preferred embodiments, there is provided a urinary catheter kit comprising: a catheter comprising a proximal end for insertion into the body and a distal end; a urine collection bag comprising an inlet adapted for fluid communication with the catheter distal end; and a catheter sleeve configured to enclose at least part of the catheter length from the proximal end to the distal end, wherein the urine collection bag and catheter sleeve comprise a trilayer laminate film, the film comprising in order: a first ethylene-vinyl acetate layer, a polyethylene layer, and a second ethylene-vinyl acetate layer, wherein the polyethylene layer of the film comprises polyethylene in a total amount of at least 80 wt. % of the polyethylene layer, and preferably the polyethylene layer is entirely made of polyethylene.

In some preferred embodiments, there is provided a urinary catheter kit comprising: a catheter comprising a proximal end for insertion into the body and a distal end; a urine collection bag comprising an inlet adapted for fluid communication with the catheter distal end; and a catheter sleeve configured to enclose at least part of the catheter length from the proximal end to the distal end, wherein the urine collection bag and catheter sleeve comprise a trilayer laminate film, the film comprising in order: a first ethylene-vinyl acetate layer, a polyethylene layer, and a second ethylene-vinyl acetate layer, wherein the polyethylene layer of the film comprises ethylene-vinyl acetate in a total amount of less than 10 wt. % of the polyethylene layer, and preferably the polyethylene layer does not comprise ethylene-vinyl acetate.

In some preferred embodiments, there is provided a urinary catheter kit comprising: a catheter comprising a proximal end for insertion into the body and a distal end; a urine collection bag comprising an inlet adapted for fluid communication with the catheter distal end; and a catheter sleeve configured to enclose at least part of the catheter length from the proximal end to the distal end, wherein the urine collection bag and catheter sleeve comprise a trilayer laminate film, the film comprising in order: a first ethylene-vinyl acetate layer, a polyethylene layer, and a second ethylene-vinyl acetate layer, wherein at least one ethylene-vinyl acetate layer of the film comprises ethylene-vinyl acetate in a total amount of at least 95 wt. % of the ethylene-vinyl acetate layer, and preferably the at least one ethylene-vinyl acetate layer is entirely made of ethylene-vinyl acetate.

In some preferred embodiments, there is provided a urinary catheter kit comprising: a catheter comprising a proximal end for insertion into the body and a distal end; a urine collection bag comprising an inlet adapted for fluid communication with the catheter distal end; and a catheter sleeve configured to enclose at least part of the catheter length from the proximal end to the distal end, wherein the urine collection bag and catheter sleeve comprise a trilayer laminate film, the film comprising in order: a first ethylene-vinyl acetate layer, a polyethylene layer, and a second ethylene-vinyl acetate layer, wherein at least one ethylene-vinyl acetate layer of the film comprises polyethylene in a total amount of less than 10 wt. % of the ethylene-vinyl acetate layer, and preferably the at least one ethylene-vinyl acetate layer does not comprise polyethylene.

In some preferred embodiments, there is provided a urinary catheter kit comprising: a catheter comprising a proximal end for insertion into the body and a distal end; a urine collection bag comprising an inlet adapted for fluid communication with the catheter distal end; and a catheter sleeve configured to enclose at least part of the catheter length from the proximal end to the distal end, wherein the urine collection bag and catheter sleeve comprise a trilayer laminate film, the film comprising in order: a first ethylene-vinyl acetate layer, a polyethylene layer, and a second ethylene-vinyl acetate layer, wherein the first and second ethylene-vinyl acetate layers of the film are each entirely made of ethylene-vinyl acetate, and the polyethylene layer of the film is entirely made of polyethylene.

In some preferred embodiments, there is provided a urinary catheter kit comprising: a catheter comprising a proximal end for insertion into the body and a distal end; a urine collection bag comprising an inlet adapted for fluid communication with the catheter distal end; and a catheter sleeve configured to enclose at least part of the catheter length from the proximal end to the distal end, wherein the urine collection bag and catheter sleeve comprise a trilayer laminate film, the film comprising in order: a first ethylene-vinyl acetate layer, a polyethylene layer, and a second ethylene-vinyl acetate layer, wherein the film comprises polyethylene in a total amount of between 5-50 wt. %, or preferably between 10-45 wt. %.

In some preferred embodiments, there is provided a urinary catheter kit comprising: a catheter comprising a proximal end for insertion into the body and a distal end; a urine collection bag comprising an inlet adapted for fluid communication with the catheter distal end; and a catheter sleeve configured to enclose at least part of the catheter length from the proximal end to the distal end, wherein the urine collection bag and catheter sleeve comprise a trilayer laminate film, the film comprising in order: a first ethylene-vinyl acetate layer, a polyethylene layer, and a second ethylene-vinyl acetate layer, wherein the film comprises ethylene-vinyl acetate in a total amount of between 50-95 wt. %, or preferably between 55-90 wt. %.

According to a fourth aspect of the disclosure, there is provided a method of manufacturing a urine collection bag comprising the steps of:
(a) Providing a front wall and a rear wall, wherein the front and rear walls comprise a trilayer film comprising in order: a first ethylene-vinyl acetate layer, a polyethylene layer, and a second ethylene-vinyl acetate layer; and
(b) Sealing the front and rear walls together to form a cavity therebetween for containing urine;
wherein the method further comprises providing an inlet for receiving urine in the front or rear wall before or after step (b).

The urine collection bag of the fourth aspect of the disclosure is preferably the urine collection bag of the first aspect of the disclosure. Statements of disclosure above for any other aspect of the disclosure may also be applied mutatis mutandis to the fourth aspect of the disclosure.

The method may comprise a further step prior to step (a) of forming the trilayer film of the front and/or rear wall. Forming the film of the front and/or rear wall may comprise: providing a first and second ethylene-vinyl acetate layer polymer composition; providing a polyethylene layer polymer composition; and co-extruding the first and second ethylene-vinyl acetate layer polymer compositions and the polyethylene layer polymer composition to provide the trilayer film, such that the film comprises in order: the first ethylene-vinyl acetate layer, the polyethylene layer, and the second ethylene-vinyl acetate layer. The polymer compositions may comprise polymer melts. Alternatively, the polymer compositions may comprise solid polymer sheets or pellets. The extrusion method may comprise multilayer cast extrusion.

Step (b) may comprise welding the front and rear walls together, preferably as described for the first aspect of the disclosure above.

The welding may employ a method that is independently selected from the group consisting of: ultrasonic welding, heat sealing, radio frequency welding, laser welding, and combinations thereof.

In some embodiments, the method comprises providing an inlet in the front or rear wall before step (b). In other embodiments, the method comprises providing an inlet in the front or rear wall after step (b). The inlet may take the form of an aperture in the front or rear wall.

The method may comprise the step of providing an attachment element in the front or rear wall for attaching the bag to a urinary catheter. The step of providing an attachment element may take place before or after step (b). In some embodiments, the attachment element comprises a mechanical fastening means. The attachment element may comprise a non-return valve to prevent flow of urine from the bag back into the catheter, in use. The non-return valve may be placed between the layers of the film. The non-return valve may be placed in an aperture comprising the inlet in the front or rear wall.

In some preferred embodiments, there is provided a method of manufacturing a urine collection bag comprising the steps of:
(a) Providing a front wall and a rear wall, wherein the front and rear walls comprise a trilayer film comprising in order: a first ethylene-vinyl acetate layer, a polyethylene layer, and a second ethylene-vinyl acetate layer, wherein the polyethylene layer of the film comprises polyethylene in a total amount of at least 80 wt. % of the polyethylene layer, and preferably the polyethylene layer is entirely made of polyethylene; and
(b) Sealing the front and rear walls together to form a cavity therebetween for containing urine;
   wherein the method further comprises providing an inlet for receiving urine in the front or rear wall before or after step (b).

In some preferred embodiments, there is provided a method of manufacturing a urine collection bag comprising the steps of:
(a) Providing a front wall and a rear wall, wherein the front and rear walls comprise a trilayer film comprising in order: a first ethylene-vinyl acetate layer, a polyethylene layer, and a second ethylene-vinyl acetate layer, wherein the polyethylene layer of the film comprises ethylene-vinyl acetate in a total amount of less than 10 wt. % of the polyethylene layer, and preferably the polyethylene layer does not comprise ethylene-vinyl acetate; and
(b) Sealing the front and rear walls together to form a cavity therebetween for containing urine;
   wherein the method further comprises providing an inlet for receiving urine in the front or rear wall before or after step (b).

In some preferred embodiments, there is provided a method of manufacturing a urine collection bag comprising the steps of:
(a) Providing a front wall and a rear wall, wherein the front and rear walls comprise a trilayer film comprising in order: a first ethylene-vinyl acetate layer, a polyethylene layer, and a second ethylene-vinyl acetate layer, wherein at least one ethylene-vinyl acetate layer of the film comprises ethylene-vinyl acetate in a total amount of at least 95 wt. % of the ethylene-vinyl acetate layer, and preferably the at least one ethylene-vinyl acetate layer is entirely made of ethylene-vinyl acetate; and
(b) Sealing the front and rear walls together to form a cavity therebetween for containing urine;
   wherein the method further comprises providing an inlet for receiving urine in the front or rear wall before or after step (b).

In some preferred embodiments, there is provided a method of manufacturing a urine collection bag comprising the steps of:
(a) Providing a front wall and a rear wall, wherein the front and rear walls comprise a trilayer film comprising in order: a first ethylene-vinyl acetate layer, a polyethylene layer, and a second ethylene-vinyl acetate layer, wherein at least one ethylene-vinyl acetate layer of the film comprises polyethylene in a total amount of less than 10 wt. % of the ethylene-vinyl acetate layer, and preferably the at least one ethylene-vinyl acetate layer does not comprise polyethylene; and
(b) Sealing the front and rear walls together to form a cavity therebetween for containing urine;
   wherein the method further comprises providing an inlet for receiving urine in the front or rear wall before or after step (b).

In some preferred embodiments, there is provided a method of manufacturing a urine collection bag comprising the steps of:
(a) Providing a front wall and a rear wall, wherein the front and rear walls comprise a trilayer film comprising in order: a first ethylene-vinyl acetate layer, a polyethylene layer, and a second ethylene-vinyl acetate layer, wherein the first and second ethylene-vinyl acetate layers of the film are each entirely made of ethylene-vinyl acetate, and the polyethylene layer of the film is entirely made of polyethylene; and
(b) Sealing the front and rear walls together to form a cavity therebetween for containing urine;
   wherein the method further comprises providing an inlet for receiving urine in the front or rear wall before or after step (b).

In some preferred embodiments, there is provided a method of manufacturing a urine collection bag comprising the steps of:
(a) Providing a front wall and a rear wall, wherein the front and rear walls comprise a trilayer film comprising in order: a first ethylene-vinyl acetate layer, a polyethylene layer, and a second ethylene-vinyl acetate layer, wherein the film comprises polyethylene in a total amount of between 5-50 wt. %, or preferably between 10-45 wt. %; and
(b) Sealing the front and rear walls together to form a cavity therebetween for containing urine;
   wherein the method further comprises providing an inlet for receiving urine in the front or rear wall before or after step (b).

In some preferred embodiments, there is provided a method of manufacturing a urine collection bag comprising the steps of:
(a) Providing a front wall and a rear wall, wherein the front and rear walls comprise a trilayer film comprising in order: a first ethylene-vinyl acetate layer, a polyethylene layer, and a second ethylene-vinyl acetate layer, wherein the film comprises ethylene-vinyl acetate in a total amount of between 50-95 wt. %, or preferably between 55-90 wt. %; and
(b) Sealing the front and rear walls together to form a cavity therebetween for containing urine;
   wherein the method further comprises providing an inlet for receiving urine in the front or rear wall before or after step (b).

### DETAILED DESCRIPTION OF THE DISCLOSURE

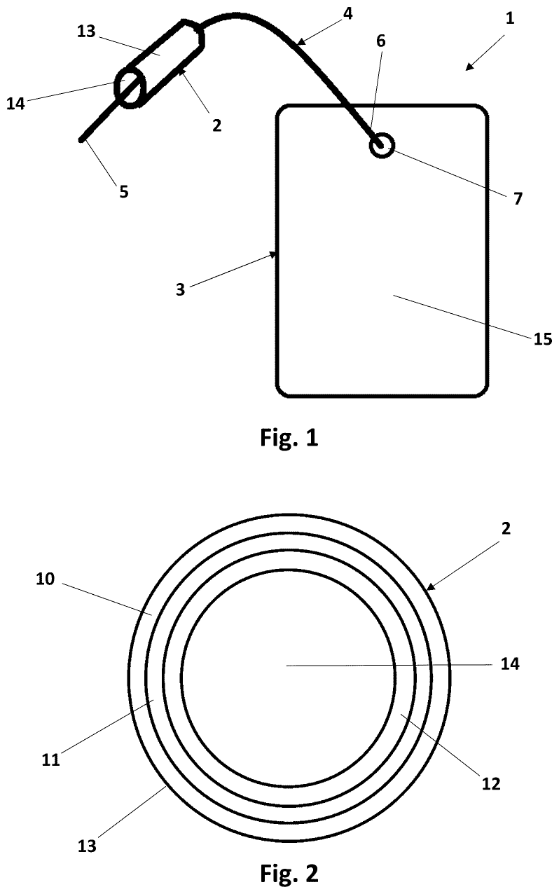

In order that the disclosure may be more clearly understood embodiments thereof will now be described, by way of example only, with reference to the accompanying drawings, of which: FIG. 1 shows a perspective view of an embodiment of a urinary catheter kit of the disclosure comprising an embodiment of a urine collection bag and catheter sleeve of the disclosure.

FIG. 2 shows a cross-sectional view through the catheter sleeve of FIG. 1.

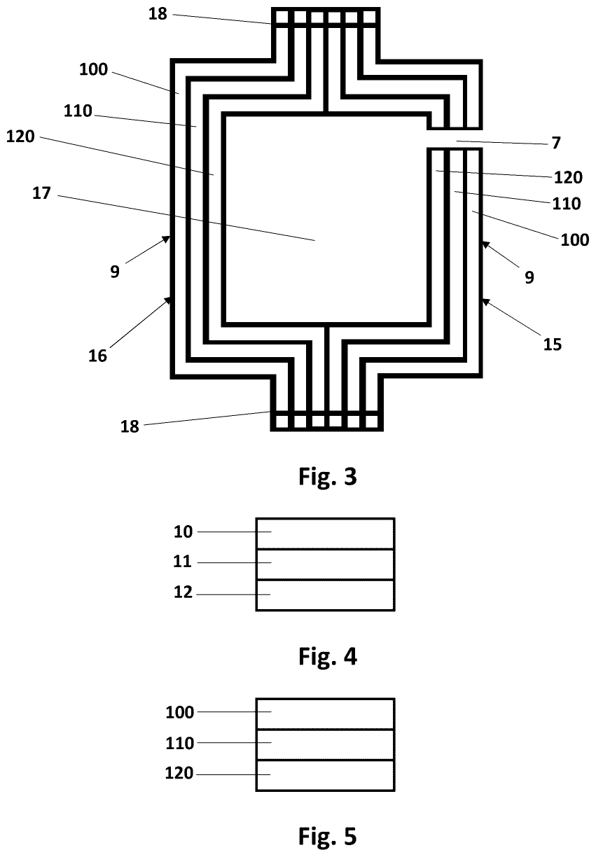

FIG. 3 shows a side cross-sectional view of the urine collection bag of FIG. 1.

FIG. 4 shows a cross-sectional view of the trilayer laminate film of the catheter sleeve of FIG. 1.

FIG. 5 shows a cross-sectional view of the trilayer laminate film of the urine collection bag of FIG. 1.

### URINARY CATHETER KIT OF THE DISCLOSURE

FIG. 1 illustrates an embodiment of a urinary catheter kit 1 of the disclosure comprising an embodiment of a catheter sleeve 2 and a urine collection bag 3 of the disclosure.

The urinary catheter kit 1 comprises an intermittent urinary catheter 4 comprising a proximal end 5 for insertion into the body and a distal end 6. The kit 1 further comprises the urine collection bag 3 which comprises an inlet 7 adapted for fluid communication with the catheter distal end 6. The distal end 6 of the catheter 4 is attached to the bag 3 at the inlet 7. The kit 1 further comprises catheter sleeve 2 which is wrapped around and encloses approximately a third of the length of the catheter 4 from the proximal 5 to the distal end 6. The catheter sleeve 2 is slidable along the length of the catheter 4 and so can be moved from the proximal end 5 to the distal end 6 and vice versa, in use.

Both the catheter sleeve 2 and the urine collection bag 3 comprise a trilayer laminate film 8,9. The film 8 of the catheter sleeve 2 and the film 9 of the bag 3 both comprise in order: a first ethylene-vinyl acetate layer 10,100, a polyethylene layer 11,110, and a second ethylene-vinyl acetate layer 12,120.

The structure of the catheter sleeve 2 is more clearly illustrated by the cross-sectional view in FIG. 2. Sleeve 2 comprises a tubular body 13 comprising the trilayer film 8. The tubular body 13 comprises a hollow lumen 14 extending longitudinally therethrough through which the catheter 4 is received, as shown in FIG. 1. The sleeve 2 is formed entirely of the trilayer film 8.

The structure of the urine collection bag 3 is more clearly illustrated by the side cross-sectional view of the bag 3 in FIG. 3. Bag 3 comprises a front wall 15 and a rear wall 16. Both the front wall 15 and the rear wall 16 comprise trilayer laminate film 9. The front wall 15 and the rear wall 16 are sealed together to form a cavity 17 of the bag 3 therebetween for holding urine. The front wall 15 and the rear wall 16 are sealed together by a weld 18 formed at their peripheries to form the cavity 17 of the bag 3. The weld 18 passes through all layers of the films 9 of the front 15 and rear walls 16 in order to hold the front 15 and rear wall 16 together.

The front wall 15 of the bag 3 further comprises the inlet 7 adapted for fluid communication with the catheter distal end 6. The inlet 7 comprises an aperture formed in the front wall 15.

Urinary catheter kit 1 is used in the conventional manner.

### Structure of Trilayer Laminate Film 8 of Catheter Sleeve 2

FIG. 4 shows a cross-sectional view of the trilayer laminate film 8 of the catheter sleeve 2.

The trilayer film 8 has a layer structure as described in Table 1 below. The order of arrangement of the layers of the film 8 are as depicted in Table 1.

TABLE 1

| | | | Layer thickness |
|---|---|---|---|
| Layer No. | Layer type | Layer materials | (μm) |
| 10 | First ethylene-vinyl | Ethylene-vinyl | 20 |
| | acetate layer | acetate: 100% | |
| 11 | Polyethylene layer | Polyethylene: | 20 |
| | | 100% | |
| 12 | Second ethylene- | Ethylene-vinyl | 20 |
| | vinyl acetate layer | acetate: 100% | |

The percentage by weight of polyethylene in film 8 is approximately 33 wt. % and the percentage by weight of ethylene-vinyl acetate in film 8 is approximately 66 wt. %.

The trilayer film 8 is produced by multilayer cast extrusion, which involves providing in polymer melt form: first and second ethylene-vinyl acetate layer polymer compositions corresponding to layers 10 and 12, and a polyethylene layer polymer composition corresponding to layer 11. The polymer compositions for the layers of the film are then co-extruded to form the trilayer film 8.

### Structure of Trilayer Laminate Film 9 of Urine Collection Bag 3

FIG. 5 shows a cross-sectional view of the trilayer laminate film 9 of the urine collection bag 3.

The trilayer film 9 has a layer structure as described in Table 2 below. The order of arrangement of the layers of the film 9 are as depicted in Table 2.

TABLE 2

| | | | Layer thickness |
|---|---|---|---|
| Layer No. | Layer type | Layer materials | (μm) |
| 100 | First ethylene-vinyl | Ethylene-vinyl | 25 |
| | acetate layer | acetate: 100% | |
| 110 | Polyethylene layer | Polyethylene: | 25 |
| | | 100% | |
| 120 | Second ethylene- | Ethylene-vinyl | 25 |
| | vinyl acetate layer | acetate: 100% | |

The percentage by weight of polyethylene in film 9 is approximately 33 wt. % and the percentage by weight of ethylene-vinyl acetate in film 9 is approximately 66 wt. %.

As for film 8 above, the trilayer film 9 is also produced by multilayer cast extrusion, which involves providing in polymer melt form: first and second ethylene-vinyl acetate layer polymer compositions corresponding to layers 100 and 120, and a polyethylene layer polymer composition corresponding to layer 110. The polymer compositions for the layers of the film are then co-extruded to form the trilayer film 9.

### Alternative Trilayer Laminate Films

Tables 3 and 4 below describe examples of alternative trilayer laminate films which could also be used in other embodiments of urine collection bags and catheter sleeves of the disclosure. Table 3 describes a trilayer film particularly suited for use in construction of a catheter sleeve of the disclosure, and Table 4 describes a film particularly suited for use in construction of a urine collection bag of the disclosure. The order of arrangement of the layers of the films are as described in the respective tables.

TABLE 3

| | | | Layer thickness |
|---|---|---|---|
| Layer No. | Layer type | Layer materials | (μm) |
| 200 | First ethylene-vinyl | Ethylene-vinyl | 15 |
| | acetate layer | acetate: 100% | |
| 210 | Polyethylene layer | Polyethylene: | 20 |
| | | 100% | |
| 220 | Second ethylene- | Ethylene-vinyl | 15 |
| | vinyl acetate layer | acetate: 100% | |

TABLE 4

| | | | Layer thickness |
|---|---|---|---|
| Layer No. | Layer type | Layer materials | (μm) |
| 300 | First ethylene-vinyl | Ethylene-vinyl | 22.5 |
| | acetate layer | acetate: 100% | |
| 310 | Polyethylene layer | Polyethylene: | 30 |
| | | 100% | |
| 320 | Second ethylene- | Ethylene-vinyl | 22.5 |
| | vinyl acetate layer | acetate: 100% | |

The percentage by weight of polyethylene in both films is approximately 40 wt. and the percentage by weight of ethylene-vinyl acetate is approximately 60 wt. %.

Tables 5 and 6 below describe two further examples of alternative trilayer laminate films which could also be used in other embodiments of urine collection bags and catheter sleeves of the disclosure.

Table 5 describes a trilayer film particularly suited for use in construction of a catheter sleeve of the disclosure, and Table 6 describes a film particularly suited for use in construction of a urine collection bag of the disclosure. The order of arrangement of the layers of the films are as described in the respective tables.

TABLE 5

| | | | Layer thickness |
|---|---|---|---|
| Layer No. | Layer type | Layer materials | (μm) |
| 400 | First ethylene-vinyl | Ethylene-vinyl | 20 |
| | acetate layer | acetate: 100% | |
| 410 | Polyethylene layer | Polyethylene: | 5 |
| | | 100% | |
| 420 | Second ethylene- | Ethylene-vinyl | 20 |
| | vinyl acetate layer | acetate: 100% | |

TABLE 6

| | | | Layer thickness |
|---|---|---|---|
| Layer No. | Layer type | Layer materials | (μm) |
| 500 | First ethylene-vinyl | Ethylene-vinyl | 40 |
| | acetate layer | acetate: 100% | |
| 510 | Polyethylene layer | Polyethylene: | 10 |
| | | 100% | |
| 520 | Second ethylene- | Ethylene-vinyl | 40 |
| | vinyl acetate layer | acetate: 100% | |

The percentage by weight of polyethylene in both films is approximately 11 wt. % and the percentage by weight of ethylene-vinyl acetate is approximately 89 wt. %.

### Results

Trilayer laminate films of the disclosure described above provide several advantages over traditional materials. The combination of layers employed confers exceptional strength, even though the films are thin—e.g. for films 8 and 9, having respective film thicknesses of 60 microns and 75 microns. The fact that such thin layers of film can be used successfully significantly reduces the amount of plastic required in manufacture of the films, which helps minimise plastic waste and contributes to environmental sustainability.

The films are also lightweight despite their high strength, which reduces unnecessary load that a user must carry when performing catheterisation. In the case of film 9, for instance, the film 9 allows for construction of a lightweight urine collection bag 3 that can still hold large amounts of urine effectively.

The films are also relatively quiet when handled and do not produce significant rustling or other disruptive sounds during movement, unlike various conventional materials previously used to make catheter kit components. This significantly enhances patient comfort and privacy, especially in the case of self-catheterisation.

The films also provide excellent resistance to discolouration when subjected to sterilisation processes. Sterilisation is a critical step to maintain the hygiene and safety of catheter kit components and the films preserve their appearance and integrity even after repeated sterilisation cycles.

The above embodiments are described by way of example only. Many variations are possible without departing from the scope of the disclosure as defined in the appended claims.

1. A urine collection bag comprising a cavity for containing urine, the bag comprising a trilayer laminate film, the film comprising in order: a first ethylene-vinyl acetate layer, a polyethylene layer, and a second ethylene-vinyl acetate layer.

2. A urine collection bag as claimed in claim 1, wherein the cavity of the bag is defined by the trilayer film.

3. A urine collection bag as claimed in any preceding claim, wherein the bag comprises a front wall and a rear wall, wherein the front and rear walls are sealed together to form the cavity of the bag therebetween, and wherein the front and rear wall comprise the trilayer film.

4. A urine collection bag as claimed in any preceding claim, wherein at least one layer of the film has a thickness of between 10-40 microns.

5. A urine collection bag as claimed in any preceding claim, wherein at least one ethylene-vinyl acetate layer has a thickness of between 10-40 microns.

6. A urine collection bag as claimed in any preceding claim, wherein the polyethylene layer has a thickness of between 10-40 microns.

7. A urine collection bag as claimed in any preceding claim, wherein the film has a total thickness of between 50-100 microns.

8. A catheter sleeve comprising a trilayer laminate film comprising in order: a first ethylene-vinyl acetate layer, a polyethylene layer, and a second ethylene-vinyl acetate layer.

9. A catheter sleeve as claimed in claim 8, wherein at least one layer of the film has a thickness of between 5-30 microns.

10. A catheter sleeve as claimed in claim 8 or 9, wherein at least one ethylene-vinyl acetate layer has a thickness of between 5-30 microns.

11. A catheter sleeve as claimed in any one of claims 8 to 10, wherein the polyethylene layer has a thickness of between 5-30 microns.

12. A catheter sleeve as claimed in any one of claims 8 to 11, wherein the film has a total thickness of between 40-70 microns.

13. A urine collection bag as claimed in any one of claims 1 to 7 or a catheter sleeve as claimed in any one of claims 8 to 12, wherein the polyethylene layer of the film comprises polyethylene in a total amount of at least 80 wt. % of the polyethylene layer, and preferably the polyethylene layer is entirely made of polyethylene.

14. A urine collection bag as claimed in any one of claims 1 to 7 or claim 13 or a catheter sleeve as claimed in any one of claims 8 to 13, wherein the polyethylene layer of the film comprises ethylene-vinyl acetate in a total amount of less than 10 wt. % of the polyethylene layer, and preferably the polyethylene layer does not comprise ethylene-vinyl acetate.

15. A urine collection bag as claimed in any one of claims 1 to 7 or claim 13 or 14 or a catheter sleeve as claimed in any one of claims 8 to 14, wherein at least one ethylene-vinyl acetate layer of the film comprises ethylene-vinyl acetate in a total amount of at least 95 wt. % of the ethylene-vinyl acetate layer, and preferably the at least one ethylene-vinyl acetate layer is entirely made of ethylene-vinyl acetate.

16. A urine collection bag as claimed in any one of claims 1 to 7 or in any one of claims 13 to 15 or a catheter sleeve as claimed in any one of claims 8 to 15, wherein at least one ethylene-vinyl acetate layer of the film comprises polyethylene in a total amount of less than 10 wt. % of the ethylene-vinyl acetate layer, and preferably the at least one ethylene-vinyl acetate layer does not comprise polyethylene.

17. A urine collection bag as claimed in any one of claims 1 to 7 or in any one of claims 13 to 16 or a catheter sleeve as claimed in any one of claims 8 to 16, wherein the first and second ethylene-vinyl acetate layers of the film are each entirely made of ethylene-vinyl acetate, and the polyethylene layer of the film is entirely made of polyethylene.

18. A urine collection bag as claimed in any one of claims 1 to 7 or in any one of claims 13 to 17 or a catheter sleeve as claimed in any one of claims 8 to 17, wherein both the first and second ethylene-vinyl acetate layers of the film have the same thickness, and optionally all three layers of the trilayer film have the same thickness.

19. A urine collection bag as claimed in any one of claims 1 to 7 or in any one of claims 13 to 18 or a catheter sleeve as claimed in any one of claims 8 to 18, wherein the film comprises polyethylene in a total amount of between 5-50 wt. %, or preferably between 10-45 wt. %.

20. A urine collection bag as claimed in any one of claims 1 to 7 or in any one of claims 13 to 19 or a catheter sleeve as claimed in any one of claims 8 to 19, wherein the film comprises ethylene-vinyl acetate in a total amount of between 50-95 wt. %, or preferably between 55-90 wt. %.

21. A urine collection bag as claimed in any one of claims 1 to 7 or in any one of claims 13 to 20 or a catheter sleeve as claimed in any one of claims 8 to 20, wherein the polyethylene layer comprises low-density polyethylene.

22. A urine collection bag as claimed in any one of claims 1 to 7 or in any one of claims 13 to 21 or a catheter sleeve as claimed in any one of claims 8 to 21, wherein the trilayer film is symmetrical.

23. A urine collection bag as claimed in any one of claims 1 to 7 or in any one of claims 13 to 22 or a catheter sleeve as claimed in any one of claims 8 to 22, wherein the trilayer film is a cast film.

24. A urinary catheter kit comprising: a catheter comprising a proximal end for insertion into the body and a distal end; a urine collection bag comprising an inlet adapted for fluid communication with the catheter distal end; and a catheter sleeve configured to enclose at least part of the catheter length from the proximal end to the distal end, wherein the urine collection bag and catheter sleeve comprise a trilayer laminate film, the film comprising in order: a first ethylene-vinyl acetate layer, a polyethylene layer, and a second ethylene-vinyl acetate layer.

25. A method of manufacturing a urine collection bag comprising the steps of:
a. Providing a front wall and a rear wall, wherein the front and rear walls comprise a trilayer barrier film comprising in order: a first ethylene-vinyl acetate layer, a polyethylene layer, and a second ethylene-vinyl acetate layer; and
b. Sealing the front and rear walls together to form a cavity therebetween for containing urine;
wherein the method further comprises providing an inlet for receiving urine in the front or rear wall before or after step (b).
